(19) United States
(12) Patent Application Publication
(10) Pub. No.: US 2016/0073916 A1
(43) Pub. Date: Mar. 17, 2016

(54) Method For Locating A Brain Activity, In Particular For Direct Neural Control

(71) Applicant: COMMISSARIAT A L'ENERGIE ATOMIQUE ET AUX ENERGIES ALTERNATIVES, Paris (FR)

(72) Inventors: Tetiana Aksenova, St Egreve (FR); Etienne Labyt, St Martin De Vinoux (FR); Ales Mishchenko, St Petersbourg (RU)

(21) Appl. No.: 14/764,695

(22) PCT Filed: Jan. 29, 2014
(86) PCT No.: PCT/IB2014/058635

§ 371 Date: Nov. 2, 2015

(30) Foreign Application Priority Data
Jan. 30, 2013 (FR) 1350783

(51) Int. Cl. A61B 5/04; A61B 5/00; A61B 5/0484

(52) U.S. Cl. CPC A61B 5/04009; A61B 5/0484; A61B 5/7246; A61B 5/742; A61B 5/04012

(57) ABSTRACT

Method for locating a brain activity, including the following steps: a) applying to a subject a first series of sensory stimuli and acquiring, by a group of sensors, respective first series of signals representative of a brain activity associated with a first task effected or imagined by the subject in response to the sensory stimuli of the first series, each sensor being sensitive to the activity of a respective region of the brain of the subject; b) applying to the subject a second series of sensory stimuli and acquiring, by the group of sensors, respective second series of signals representative of a brain activity associated with a second task, different from the first task, effected or imagined by the subject in response to the sensory stimuli of the second series; and c) constructing, for each sensor, a multidimensional variable representative of the corresponding first and second series of signals, and determining a coefficient of correlation between the multidimensional variable and an observation vector representative of the first and second sensory stimuli.

The invention relates to a method for locating brain activity of a subject, notably by magnetoencephalogy. The invention applies in particular to the field of direct neural control.

Direct neural control (BCI, “brain-computer interface”) makes it possible to establish a communication between a user and a machine (typically a computer) through neural signals deriving from the brain activity of a subject without the use of the muscular pathway, which constitutes a real hope for people suffering from serious paralyses.

Non-intrusive direct neural control systems use, more often than not, electroencephalography (EEG) as the method for acquiring brain activity. Thus, a certain number of electrodes are placed on the surface of the cranium in order to measure therein an electrical activity reflecting the brain activity of the subject. Other techniques, more efficient but also more intrusive, exploit electrocorticographic signals (ECoG), sampled at the surface of the cortex, even signals sampled by deep electrodes. Magnetoencephalogy (MEG) is a non-intrusive technique, whose use in direct neural control is conceptually interesting, because the magnetic signals undergo little or no distortion when they are propagated through the cranium. Its main drawback, which in practice limits it to experimental applications, is the insufficient miniaturization of the magneto encephalographic sensors.

Whatever the brain activity acquisition method used, the principle on which direct neural control is based generally consists in associating one or more mental tasks (action imagined by the subject) with one or more actions performed by an effecter. For example, the imagination of the movement of the right hand can be associated with the displacement to the right of a cursor.

The inclusion of the spatial information conveyed by the neural signals is important in producing this association. In effect, the performing of different mental tasks activates different regions of the brain, or the same regions but in a different way. To preserve this spatial information to the greatest possible extent, a large number of sensors (up to a hundred or so) are in most cases used. This approach presents a number of drawbacks: a nuisance for the user, a lengthy preparation time, and a high computational cost. Furthermore, certain types of treatments show limitations when the number of sensors increases (for example, over-learning effects are observed). Thus, techniques have been developed to determine the optimal placements on the cranium or on the surface of the cortex of a subject in which to situate a number of sensors that is as limited as possible. For example, the article by A. Barachant, T. Aksenova, and S. Bonnet, “Filtrage spatial robuste à partir d'un sous-ensemble optimal d'électrodes en BCI EEG” [Robust spatial filtering based on an optimal subset of electrodes in EEG BCI] GRETSI 2009, 8-11 Sep. 2009, describes an ascending selection method (that is to say one in which an optimal set of sensors is constructed progressively), based on a criterion of multiple correlation of the log-variants of the EEG signals after frequency filtering.

The French patent application 12 56292, filed on Jun. 26, 2012, describes a method for locating brain activity of a subject involved in a task, using in particular magnetoencephalogy. This method is based on the computation of a determination coefficient expressing the correlation between the signals obtained from a sensor (consisting in particular of a magnetometer and of a pair of gradiometers) and an observation vector indicative of the presence of a sensory stimulus which triggers performance of the task by the subject. The sensors that exhibit the highest determination co-efficients represent the regions of the brain that are most active, which can be preferentially used to produce a direct neural control.

The present inventors have appreciated that this method, like all the techniques known from the prior art and aiming to establish a correlation between brain activity (notably cortical) and a task performed in response to a sensory stimulus, present the drawback of detecting certain regions of the brain which in reality prove non-specific to the task concerned. The signals coming from these regions are therefore spurious signals, whose inclusion is detrimental to the effectiveness of the neural control. A study has made it possible to determine that these non-specific regions are not activated by the task studied but by the perception of the sensory stimulus; they are therefore primarily visual or auditory areas of the cortex, depending on whether the stimulus is a visual signal or a sound.

The invention aims to overcome this drawback of the prior art by allowing for a better discrimination between regions of the brain that are specific and non-specific to the task concerned.

According to the invention, this aim is achieved by having the subject under study (generally a human being, but in certain cases it may be an erect animal) perform not one, but (at least) two mutually different successive tasks, in response to respective sensory stimuli. The joint inclusion of the neural signals acquired during the performance of the different tasks makes it possible to dispense with the influence of the non-specific brain regions, activated by the perception of the stimulus rather than by the tasks themselves. The two sensory stimuli will be of the same nature—for example both visual or both auditory. Preferably, the two tasks performed will correspond to movements (real or imaginary) of a right limb of the body of the subject and of the corresponding left limb.

Thus, a subject of the invention is a method for locating a brain activity, comprising the following steps:

a) applying to a subject a first series of sensory stimuli and acquiring, by means of a set of sensors, first respective series of signals representative of a brain activity associated with a first task performed or imagined by said subject in response to the sensory stimuli of said first series, each said sensor being sensitive to the activity of a respective region of the brain of said subject;

b) applying to said subject a second series of sensory stimuli and acquiring, by means of said set of sensors, second respective series of signals representative of a brain activity associated with a second task, different from said first task, performed or imagined by said subject in response to the sensory stimuli of said second series; and

c) for each said sensor, constructing a multidimensional variable representative of the first and the second corresponding series of signals, and determining a correlation co-efficient between said multidimensional variable and an observation vector representative of said first and second sensory stimuli.

According to different embodiments of the invention:
Said first task can correspond to a movement of a right limb of the body of said subject and said second task can correspond to a movement of a left limb, or vice versa. More particularly, said first task can correspond to a movement of a right limb of the body of said subject and said second task to a symmetrical movement of the corresponding left limb, or vice versa.
Said step c) can comprise the concatenation of said first and second series of signals with change of sign of one of them.
Said sensory stimuli of said first and second series can be of the same nature.
Said step c) can comprise the production of a time-frequency analysis of said series of signals, in return for which said multidimensional variable can be a matrix.
Said step c) can comprise an operation of standardization and centering of said series of signals.
Said sensors can be magneto encephalographic sensors, and in particular each of said sensors can comprise a pair of gradiometers arranged to acquire two distinct spatial components of a gradient of a magnetic field generated by the brain of said subject.
The method can also comprise a display step d), during which values indicative of the correlation co-efficients determined for each said sensor are projected onto a three-dimensional model of a cortical surface, and an interpolation of said values is produced between different points of a meshing of said surface.

Another subject of the invention is a method for locating brain activity sensors for direct neural control comprising:
a step of locating a brain activity, implemented by a method as defined above; and
a step of determination of optimal locations of said brain activity sensors as a function of the results of said step of locating a brain activity.

Other features, details and advantages of the invention will emerge on reading the description given with reference to the attached drawings given by way of example and which represent, respectively:

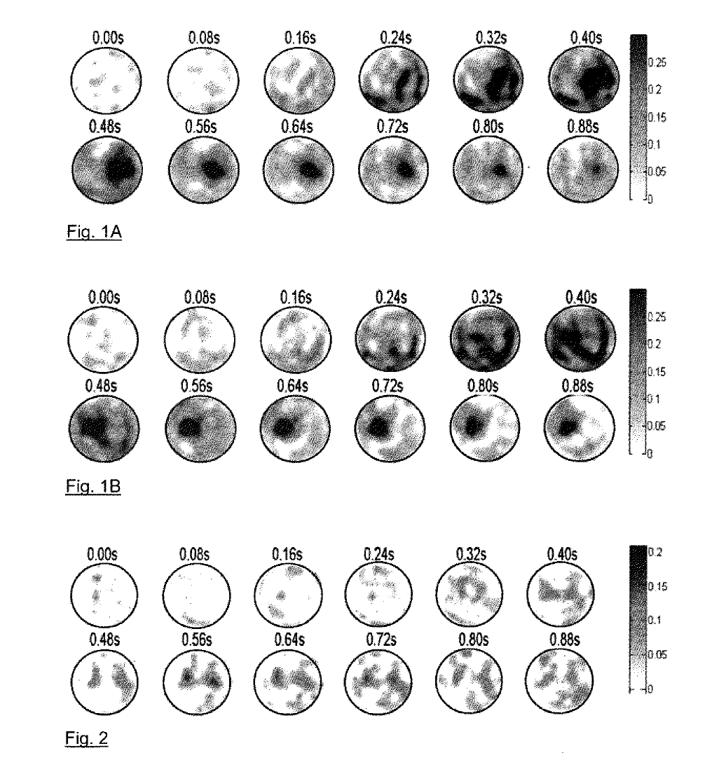

FIGS. 1A and 1B, maps of the correlation coefficients between a visual stimulus (OK) and magneto encephalographic signals acquired on a subject who, in response to this stimulus, imagines performing a movement of the left index and of the right index, respectively; and

FIG. 2, maps of correlation co-efficients obtained by a method according to an embodiment of the invention, jointly considering the magneto encephalographic signals acquired mapped to the two tasks considered.

As a nonlimiting example, the invention will be described with reference to a particular embodiment, in which the signals representative of a brain activity are acquired by means of magneto encephalographic sensors consisting of two gradiometers sensitive to components, mutually orthogonal and parallel to the surface of the cranium, of the gradient of a magnetic field generated by the cerebral cortex of the subject. In this example, the stimulus is of visual type and the two tasks performed by the subject consist in imagining a striking movement of the left or right index, respectively.

For the first task performed (imaginary movement of the left index), for each sensor and for each visual stimulus a signal is acquired that is representative of a brain activity of the subject; since in general each sensor comprises a plurality of individual sensors (in this case, two gradiometers), the signal exhibits a plurality of components. A time-frequency analysis makes it possible to represent this signal in vector form: x (tᵢ+τ)=[x¹_{f1}(tᵢ+τ) . . . x¹_{fM}(tᵢ+τ) . . . x^{Nc}_{f1}(tᵢ+τ) . . . x^{Nc}_{fM}(tᵢ+τ)]^{T} where f1-fM are spectral components of the signal, the exponent with a value of between 1 and N_{c} identifies the components of the signal originating from the different individual sensors (here: N_{C}=2), tᵢ is the instant at which the ith stimulus is administered and τ the acquisition time (time elapsed since the instant tᵢ). The dimension of the vector variable x is therefore N_{c}M.

This operation is repeated a plurality (N>1) of times, and the vectors x that are thus obtained are used to construct the matrix variable X defined as follows:

$$X = ( \begin{matrix}1 & {x}_{f 1}^{1} ( {t}_{1} + τ ) & {x}_{f 2}^{1} ( {t}_{1} + τ ) & … & {x}_{f 1}^{2} ( {t}_{1} + τ ) & {x}_{f 2}^{2} ( {t}_{1} + τ ) & … \\ 1 & {x}_{f 1}^{1} ( {t}_{2} + τ ) & {x}_{f 2}^{1} ( {t}_{2} + τ ) & … & {x}_{f 1}^{2} ( {t}_{2} + τ ) & {x}_{f 2}^{2} ( {t}_{2} + τ ) & … \\ … & … & … & … & … & … & … \\ 1 & {x}_{f 1}^{1} ( {t}_{N} + τ ) & {x}_{f 2}^{1} ( {t}_{N} + τ ) & … & {x}_{f 1}^{2} ( {t}_{N} + τ ) & {x}_{f 2}^{2} ( {t}_{N} + τ ) & …\end{matrix} )$$

Also defined is the observation vector y(t), which has the value 1 during the administration of a stimulus triggering said first task, and 0 otherwise: y=(y(t₁) y(t₂) . . . y(t_{N}))^{T}.

It will be recalled that xⁱ_{ƒk}(tⱼ+τ) represents the spectral component in the frequency band fₖ of the gradiometer i measured at time τ following instant tⱼ of recording of the observation variable y(t).

To compute the correlation coefficient R(τ), a linear regression of y relative to X is first of all performed, by writing:

$$\hat{y} ( t ) = {b}_{0} + {\sum }_{i = 1}^{M} {b}_{i}^{1} {x}_{fi}^{1 i} ( t + τ ) + {\sum }_{i = 1}^{M} {b}_{i}^{2} {x}_{fi}^{2} ( t + τ )$$

in which the vector can be obtained by the least squares method, in which case b=(X^{T}X)⁻¹X^{T}y

Then, the following formula is applied:

$${R}^{2} ( τ ) = 1 - \frac{\sum {( y ( t ) - \hat{y} ( t ) )}^{2}}{\sum {( y ( t ) - \overline{y} )}^{2}}$$

FIG. 1A shows maps of the correlation co-efficient R(τ) that is thus obtained for different values of the time τ. FIG. 1B shows maps obtained in a similar manner, but for a second task consisting in imagining a striking movement of the right index. In these figures, it can be seen significant correlations in the posterior cortex (visual area of the cortex—represented in the upper part of each image), when τ=0.08 s. This correlation corresponds to the perception of the visual stimulus by the subject. It is therefore unrelated to the correlation that is wanted to be revealed, linked with the performance of the task by the subject. This “useful” correlation is located facing motive, and not visual, regions of the cortex. These motive regions appear in the form of spot dark areas in FIGS. 1A and 1B, for τ>0.48 s.

Hereinbelow, X_{L} and X_{R} will be used to designate the matrices X corresponding to the examples illustrated in FIGS. 1A and 1B, respectively. Thus, X_{L} corresponds to an imaginary movement of the left index, whereas X_{R} corresponds to an imaginary movement of the right index. Furthermore, Y_{L} and Y_{R} will be used to designate the observation vectors y corresponding to the cases illustrated in FIGS. 1A and 1B.

Each matrix (X_{R} or X_{L}) is centered and standardized as follows:
the rows i are identified which correspond to y(tᵢ)=0, which makes it possible to construct a submatrix X_{R} (respectively X_{L}), comprising only these rows i;
the average value of each column of this submatrix is determined, which makes it possible to have a row matrix;
term by term, this row matrix is subtracted from each row of the matrix X_{R} (respectively X_{L});
the variance of each column of the matrix X′_{R} (respectively X′_{L}) that is thus obtained is determined, which makes it possible to have a row matrix representing the variance of each column;
each term of a column of the matrix X′_{R} (respectively X′_{L}) is divided by the corresponding term in the row matrix (standardization by the variance).

This makes it possible to dispense with the “physiological variance”, that is to say a temporal drift of the signals measured during the series of acquisitions. This optional step is not necessary when the acquisitions are close together, notably when the acquisitions are interlaced.

A composite matrix X_{C} is then established, obtained by concatenating the matrices X_{R} and −X_{L}:

$$Xc = [ \begin{matrix}{X}_{R} \\ - {X}_{L}\end{matrix} ]$$

or, more explicitly:

$$Xc = [ \begin{matrix}1 & & {x}_{f 1}^{1 R} ( {t}_{1} + τ ) & & {x}_{f 2}^{1 R} ( {t}_{1} + τ ) & & … & & {x}_{f 1}^{2 R} R ( {t}_{1} + τ ) & & {x}_{f 2}^{2 R} ( {t}_{1} + τ ) & & … \\ 1 & & {x}_{f 1}^{1 R} ( {t}_{2} + τ ) & & {x}_{f 2}^{1 R} ( {t}_{2} + τ ) & & … & & {x}_{f 1}^{2 R} ( {t}_{2} + τ ) & & {x}_{f 2}^{2 R} ( {t}_{2} + τ ) & & … \\ … & & … & & … & & … & & … & & … & & … \\ 1 & & {x}_{f 1}^{1 R} ( {t}_{N} + τ ) & & {x}_{f 2}^{1 R} ( {t}_{N} + τ ) & & … & & {x}_{f 1}^{2 R} ( {t}_{N} + τ ) & & {x}_{f 2}^{2 R} ( {t}_{N} + τ ) & & … \\ - 1 & & - {x}_{f 1}^{1 L} ( {t}_{1}^{′} + τ ) & & - {x}_{f 2}^{1 L} ( {t}_{1}^{′} + τ ) & & … & & - {x}_{f 1}^{2 L} ( {t}_{1} + τ ) & & - {x}_{f 2}^{2 L} ( {t}_{1} + τ ) & & … \\ - 1 & & - {x}_{f 1}^{1 L} ( {t}_{2} + τ ) & & - {x}_{f 2}^{1 L} ( {t}_{2} + τ ) & & … & & - {x}_{f 1}^{2 L} ( {t}_{2} + τ ) & & - {x}_{f 2}^{2 L} ( {t}_{2} + τ ) & & … \\ … & & … & & … & & … & & … & & … & & … \\ - 1 & & - {x}_{f 1}^{1 L} ( {t}_{M}^{′} + τ ) & & - {x}_{f 2}^{1 L} ( {t}_{N} + τ ) & & … & & - {x}_{f 1}^{2 L} ( {t}_{N} + τ ) & & - {x}_{f 2}^{2 L} ( {t}_{N} + τ ) & & …\end{matrix} ]$$

Similarly, a composite observation vector y_{c} is established, which is the concatenation of the vectors y_{L} and y_{R},

$$yc = [ \begin{matrix}{y}_{R} \\ {y}_{L}\end{matrix} ]$$

Then, a correlation coefficient R_{c}(τ) of X_{C} with y_{c} is determined as previously indicated. FIG. 2 shows maps of this “composite” correlation co-efficient R_{c}(τ) for different values of τ. An improved spatial resolution is observed in relation to the cases of FIGS. 1A and 1B, notably between τ=0.4 and 0.72 s. Above all, the correlations in the visual regions of the cortex have disappeared.

The vectors y_{R} and y_{L} can be independent of one another, but generally of the same size, or of comparable sizes.

Using the method described above, the end result is a correlation co-efficient for each measurement point (sensor) as a function of the time τ between the stimulus and the measurement. Correlation co-efficient values are then available according to a spatial meshing defined by the positioning of the sensors. It is possible to work on the basis of this meshing to produce a projection of said values onto the surface of the cortex. For this, the surface of the cortex is obtained, for example from MRI measurements, and is then modeled. The meshing formed by the different sensors is then realigned to this model, for example by using stereotaxic reference frames which are visible in MRI, notably pellets of gadolinium salt arranged on the head of the patient.

From the determination co-efficient values, a projection onto the model of the cortical surface is produced, the value assigned to each element of said cortical surface being derived from an interpolation between different points of the meshing, for example the three closest neighbors, the weighting criterion being a distance.

In certain applications only the absolute values of the correlation co-efficients are considered, their signs being of no interest. Thus, preferably, sensors intended to produce a direct neural control will preferably be placed mapped to the regions of the cortex exhibiting the highest correlation co-efficients (as absolute value) with the tasks used for the control. It should be noted that these sensors can be different from those used for the locating of the brain activity. For example, magneto encephalographic sensors can be used to locate the brain (cortical) activity in accordance with the invention and ECoG electrodes can be used for the direct neural control.

The invention is not limited to the embodiment described above; in effect, a number of variants can be envisaged. For example:
Still in the context of an embodiment using magnetoencephalogy, the sensors can be of different type, and notably comprise a magnetometer instead of, or in addition to, gradiometers; similarly, a component of the magnetic field at right angles to the surface of the cranium can also be measured.
Other techniques for detecting and measuring the brain activity can be used, such as electrocorticography or electroencephalography.
The two tasks concerned need not correspond to symmetrical movements of the body of the subject. They may also be movements of different limbs (for example, movement of an arm and of a leg), situated on the same side or on opposite sides of the body, or even tasks of a different nature, not corresponding (or of which one does not correspond) to a real or imaginary movement; for example, a task may consist in imagining a color.
The centering and the standardization of the series of signals are advantageous, but not essential. Furthermore, different processing operations from those described can be applied to the signals in order to determine the correlation co-efficients.
The method can be used with a single sensor, if only the degree of activation of a specific region of the brain upon the performance of a task is to be studied.
The invention also accepts applications other than direct neural control, for example fundamental research in neurosciences.

1. A method for locating a brain activity, comprising the following steps:
a) applying to a subject a first series of sensory stimuli and acquiring, by means of a set of sensors, first respective series of signals representative of a brain activity associated with a first task performed or imagined by said subject in response to the sensory stimuli of said first series, each said sensor being sensitive to the activity of a respective region of the brain of said subject;
b) applying to said subject a second series of sensory stimuli and acquiring, by means of said set of sensors, second respective series of signals representative of a brain activity associated with a second task, different from said first task, performed or imagined by said subject in response to the sensory stimuli of said second series; said sensory stimuli of said first and said second series being of the same nature,
wherein the method comprises the step c) comprising in
for each said sensor, constructing a multidimensional variable representative of the first and the second corresponding series of signals, and determining a correlation co-efficient between said multidimensional variable and an observation vector representative of said first and second sensory stimuli,
said step c) comprising the concatenation of said first and second series of signals with change of sign of one of them.

2. The method as claimed in claim 1, in which said first task corresponds to a movement of a right limb of the body of said subject and said second task corresponds to a movement of a left limb, or vice versa.

3. The method as claimed in claim 2, in which said first task corresponds to a movement of a right limb of the body of said subject and said second task corresponds to a symmetrical movement of the corresponding left limb, or vice versa.

4. The method as claimed in claim 1, in which said step c) comprises the production of a time-frequency analysis of said series of signals, in return for which said multidimensional variable is a matrix.

5. The method as claimed in claim 1, in which said step c) comprises an operation of standardization and centering of said series of signals.

6. The method as claimed in claim 1, in which said sensors are magnetoencephalographic sensors.

7. The method as claimed in claim 6, in which each said sensor comprises a pair of gradiometers arranged to acquire two distinct spatial components of a gradient of a magnetic field generated by the brain of said subject.

8. The method as claimed in claim 1, also comprising a display step d), during which values indicative of the correlation coefficients determined for each said sensor are projected onto a three-dimensional model of a cortical surface, and an interpolation of said values is produced between different points of a meshing of said surface.

9. A method for locating brain activity sensors for direct neural control comprising:
a step of locating a brain activity, implemented by a method as claimed in claim 1; and
a step of determination of optimal locations of said brain activity sensors as a function of the results of said step of locating a brain activity.
